(19) United States
(12) Patent Application Publication
(10) Pub. No.: US 2010/0249898 A1
(43) Pub. Date: Sep. 30, 2010

(54) Stent Graft

(75) Inventors: Adrian Gale, San Francisco, CA (US); Jenine S. Vinluan, Petaluma, CA (US); Jia Hua Xiao, Santa Rosa, CA (US); Michael Francis, Loughborough (GB); Frank Yang, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/409,759

(22) Filed: Mar. 24, 2009

(51) Int. Cl. A61F 2/82

(52) U.S. Cl. 623/1.13

(57) ABSTRACT

A stent graft system includes a first stent graft having a first end and a second end opposing the first end. The system further includes a second stent graft in fluid communication with the first stent graft. The second stent graft defines a lumen a third end in fluid communication with the second end. The second stent graft further includes a fourth end opposing the third end, and at least a first sleeve and second sleeve extending radially therefrom and in fluid communication with the lumen, The first sleeve is radially offset from the second sleeve. Each of the first sleeve and second sleeve comprises a flexible material configured to evert between a first position such that the sleeve is positioned within the lumen and a second position such that the sleeve is positioned exterior to the lumen. The system further includes a third stent graft.

### TECHNICAL FIELD

The present invention relates generally to the field of implantable medical devices. More particularly, the invention relates to an endoluminal prosthetic assembly and method with enhanced perfusion.

### BACKGROUND

Aortic aneurysms are bulges or sacs that form in the aorta resulting from the vessel wall losing its elasticity. In certain cases, the force of normal blood pressure in the aneurysm may lead to the rupture of the vessel. Aneurysms are most commonly the result of fatty deposits on the vessel wall but may also result from other causes that weaken the vessel wall, including heredity, trauma, and disease. Abdominal aortic aneurysms (AAA) form in the portion of the aorta that extends through the abdomen and thoracic aortic aneurysms (TAA) form in the portion of the aorta located within the thoracic cavity.

A number of methods and devices for treating aneurysms in the aorta, either AAA or TAA have been developed. A historically standard treatment is conventional open surgery, which is performed to replace the section of the vessel where the aneurysm has formed. The aneurysm is accessed by a surgeon through an incision in the abdomen. The portion of the blood vessel where the aneurysm has formed is shut off from the main portion of the blood vessel and then replaced with a synthetic graft. Surgery is performed under general anesthesia and takes three to four hours to complete. Following the surgery, the patient may spend time in an intensive care unit and may remain in the hospital for several days.

For several reasons, including current physical condition of the patient, some patients are not good candidates for such open surgery. Due to the highly invasive nature of the open procedure, some patients may not wish to undergo the treatment. These patients have to live with the continued risk of aneurysm rupture. Thus, alternative methods of treating an AAA or TAA are desirable.

One alternative treatment is a technique known as endovascular stent grafting. In this procedure, a stent graft is placed inside the vessel affected by the aneurysm in order to reinforce the weakened vessel wall, thereby reducing strain on the vessel wall to reduce the chance of rupture of the aneurysm. The stent graft is guided to the area of the aneurysm using a delivery catheter, typically via the femoral artery and the iliac artery into the aorta. A sheath on the catheter is retracted or released allowing the self-expanding stent graft to expand and be deployed and fixed into position.

Stent grafts for use in abdominal aortic aneurysms typically include a support structure supporting woven or interlocked graft material. Examples of woven graft materials are woven polymer materials, e.g., Dacron® material. Interlocked graft materials include knit, stretch, and velour materials. Expanded polytetrafluoroethylene (ePTFE) is also sometimes used. The graft material is secured to the inner or outer diameter of the support structure, which supports the graft material and/or holds it in place against a vessel wall. The stent graft is secured to a vessel wall above and below the aneurysm. A proximal spring stent of the stent graft can be located above the aneurysm to provide a radial force to engage the vessel wall and seal the stent graft to the vessel wall. In other examples, a stent graft anchor is utilized to affix the stent graft to the wall of a vessel.

In the event of an aneurysm that is near or contains an ostium of a branch vessel, stent grafts can be difficult to place while preserving appropriate perfusion through the branch vessel. For example, for a AAA aneurysm near or involving the ostia of the renal arteries, it is desirable to treat the aneurysm while maintaining perfusion of the kidneys. Prior art solutions have included bifurcated stent grafts and aorto-uni-iliac (“AUI”) designs. However, these designs are less than optimal for some applications. Additionally, it would be desirable to have a stent graft system that is easy to place.

It would be desirable to overcome the above disadvantages.

### SUMMARY OF THE INVENTION

One aspect according to the present invention provides a stent graft system includes a first stent graft having a first end and a second end opposing the first end. The system further includes a second stent graft in fluid communication with the first stent graft. The second stent graft defines a lumen and includes a third end in fluid communication with the second end and a fourth end opposing the third end. The system further includes at least a first sleeve extending radially therefrom and in fluid communication with the lumen, and a third stent graft in fluid communication with the second stent graft, the third stent graft including a fifth end in fluid communication with the fourth end. The first sleeve includes a flexible material configured to evert between a first position such that the first sleeve is positioned within the lumen and a second position such that the first sleeve is positioned exterior to the lumen.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIG. 1 is a schematic side view of a stent graft system deployed within a vessel, in accordance with one aspect of the invention;

FIG. 2A is a schematic side view of a stent graft system deployed within a vessel, in accordance with one aspect of the invention;

FIG. 2B is a schematic side view of a stent graft system deployed within a vessel, in accordance with one aspect of the invention;

FIG. 2C is a schematic side view of a stent graft system deployed within a vessel, in accordance with one aspect of the invention;

FIG. 3 is a flowchart of the steps of deploying a stent graft system, in accordance with one aspect of the invention.

### DETAILED DESCRIPTION

Embodiments according to the invention will now be described by reference to the figures wherein like numbers refer to like structures. The terms “distal” and “proximal” are used herein with reference to the direction of blood flow from the heart in using the stent graft system in the vasculature: “distal” indicates an apparatus portion distant from, or a direction away from the heart and “proximal” indicates an apparatus portion near to, or a direction closest to the heart.

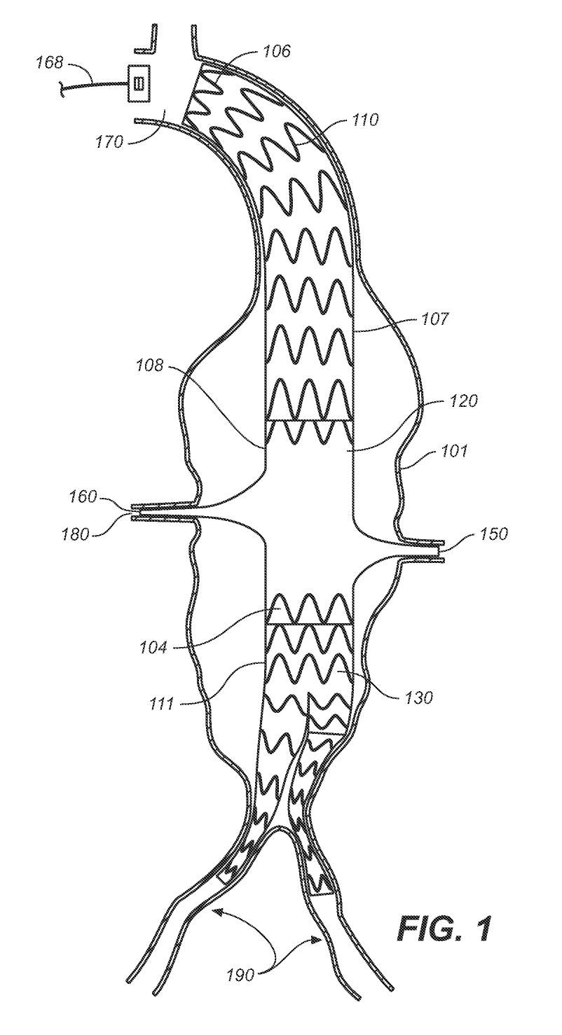

FIG. 1 illustrates one embodiment of a stent graft system 100 deployed within a vessel near an aneurysm 101, in accordance with one aspect of the invention. Stent graft system 100 includes a first stent graft 110, a second stent graft 120, and a third stent graft 130. First stent graft 110 is in fluid communication with second stent graft 120, and second stent graft 120 is in fluid communication with third stent graft 130. FIG. 1 illustrates the stent graft system deployed near the intersection of the aorta 170 and renal arteries 180 and terminating in the iliac arteries 190, although other locations are envisioned. For example, stent graft system 100 could be deployed near the celiac artery or superior mesenteric artery, or any other abdominal aortic vessel. In the embodiment illustrated in FIG. 1, the first stent graft is a TAA stent graft. In the embodiment illustrated in FIG. 1, third stent graft 130 is a bifurcated stent graft. In other embodiments, third stent graft 130 can be an aorto-uni-iliac (“AUI”) stent graft. In yet other embodiments, the third stent graft 130 can be a substantially cylindrical stent graft. Any of the first stent graft, second stent graft, and third stent graft can be either self-expanding or balloon expanding stent grafts, or any appropriate combination of self-expanding and balloon-expandable.

Second stent graft 120 includes at least a first sleeve 150 extending radially therefrom. The second stent graft 120 includes a main graft section 155, and the main graft section 155 is in fluid communication with the first sleeve 150. FIG. 1 illustrates a second sleeve 160, also extending radially from and in fluid communication with the second stent graft 120. The first sleeve, in one embodiment, comprises a flexible material configured to evert between a first position where the first sleeve 150 is positioned within the main graft section 155 and a second position such that the first sleeve 150 is positioned on an exterior of the main graft section. In one embodiment, the first sleeve when it is positioned exterior of the main graft section is substantially within a side vessel, such as the renal arteries, or other aortic sleeve vessel.

First stent graft 110 includes a first end 106 and second end 107. Second stent graft 120 includes a third end 108 and fourth end 109. Third stent graft includes at least a fifth end 111. Second end 107 and third end 108 are in fluid communication and affixed when deployed. Fourth end 109 and fifth end 111 are in fluid communication and affixed to each other when deployed. In one embodiment, when deployed, the third end 108 is positioned within a lumen defined by the second end 107 and fixed thereto by an interference fit between the pieces, and the fifth end 111 is positioned within the lumen defined by the fourth end 109 and fixed thereto by an interference fit between the pieces. In another embodiment, when deployed, the fourth end 109 is positioned within the lumen defined by the fifth end 111 and the second end 107 in positioned within the lumen defined by the third end 108. The first stent graft 110 and second stent graft 120, as well as the second stent graft 120 and third stent graft 130, can be affixed to each other to maintain their relative positions using appropriate techniques, such as interfering expandable rings, stent graft anchors, or physical contact between stent frameworks of the associated stent grafts.

Each of first stent graft 110, second stent graft 120, and third stent graft 130 includes a support structure, such as a stent framework, as well as a woven graft material supported by the support structure. The support structure is optimally manufactured from a biocompatible material, such as stainless steel, nitinol, tantalum, ceramic, nickel, titanium, aluminum, polymeric materials, MP35N, stainless steel, titanium ASTM F63-83 Grade 1, niobium, high carat gold K 19-22, combinations of the above, and the like. Positioning of the stent grafts discussed herein may be improved by ensuring that the support structure is fluoroscopically opaque. The cross-sectional shape of the finished support structures may be circular, ellipsoidal, rectangular, hexagonal, square, or other polygon, depending on the size and shape of the vessel across which the system is implanted. The woven graft material can include one or more suitable implantable materials having good tensile strength, such as a material suitable for resisting expansion when the force associated with blood pressure is applied to its tubular configuration after completion of the stent grafting procedure. The graft material may be a suitable biocompatible plastic, such as implantable quality woven polyester. In some embodiments, graft material includes components made of collagen, albumin, an absorbable polymer, or biocompatible fiber. Alternatively, graft material is one or more suitable metallic, plastic, or non-biodegradable materials. In certain embodiments, the sleeve 150 may be coated with a lubricious coating, such as a biocompatible coating. For example, the sleeve 150 may be coated with a silicone lubricious coating to ease in eversion and deployment from within the second stent graft 120 to the exterior of the second stent graft. Other coatings, such as polytetrafluroethylene (PTFE), or a hydrophilic coating are also envisioned.

In one embodiment, the support structure of the second stent graft 120 has a stent pattern on the graft material that is interrupted in the graft section near the ostium for the sleeve 150 or the artery so as to reduce inducing turbulence in the blood flow. In other embodiments, the support structure in the second stent graft 120 extends along the length and circumference of the second stent graft without interruption. Alternatively, second stent graft 120 can include two support structures, with a first support structure located near the third end, and the second support structure located near the fourth end such that the first support structure is offset from the second support structure and the first support structure does not contact the second support structure.

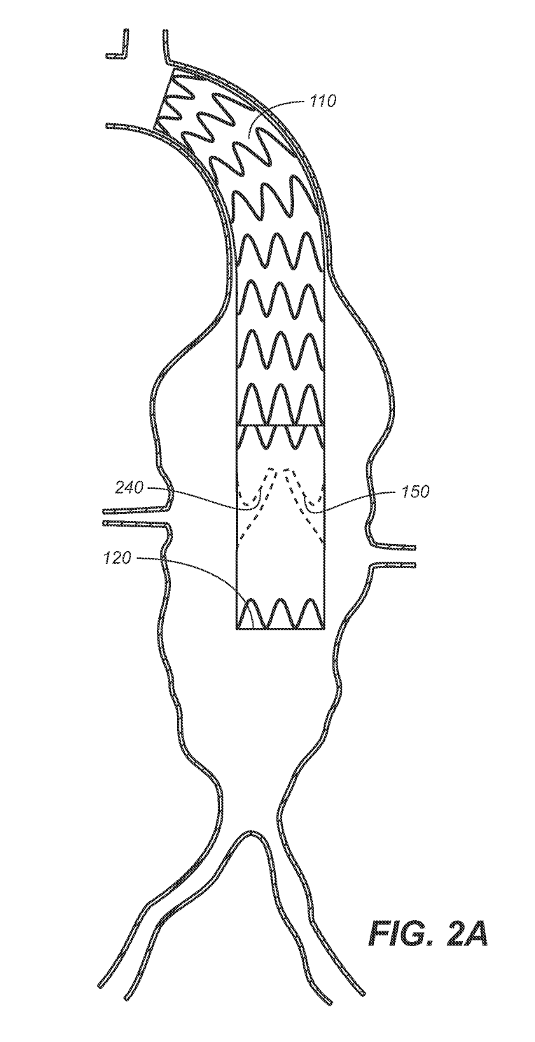

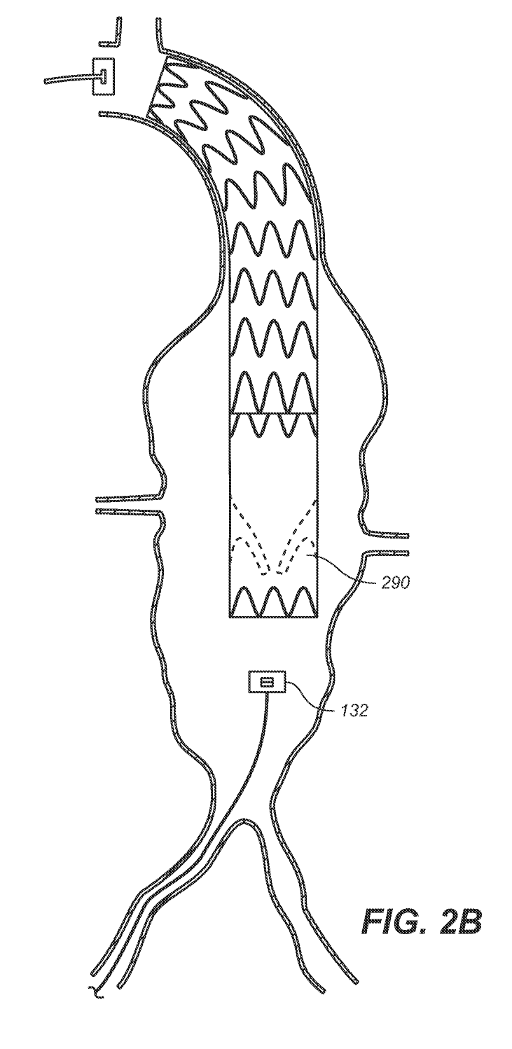

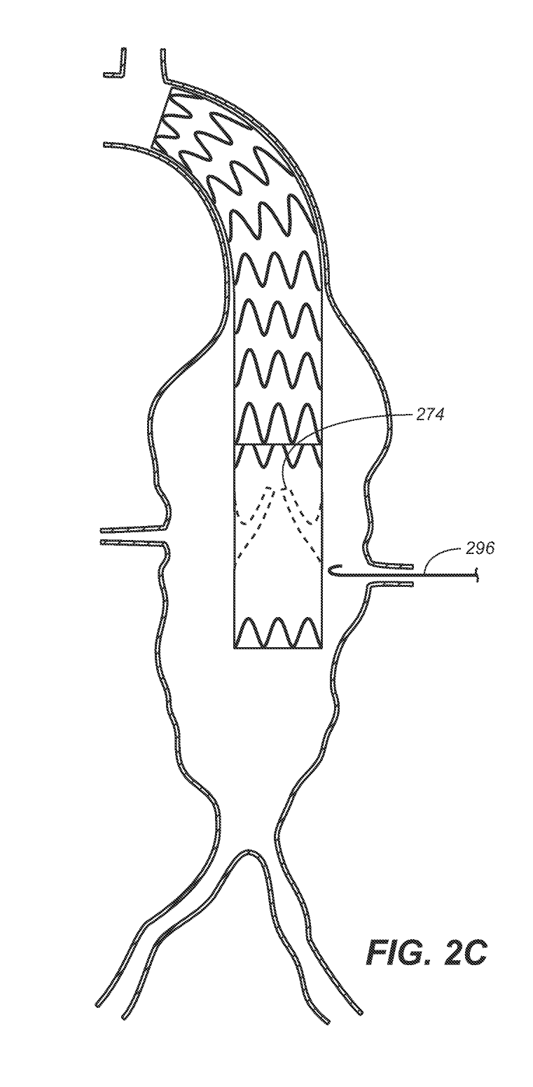

In one embodiment, the system further includes at least a first plunger sized to extend the first sleeve from inside main graft section 155 to the exterior of the main graft section. In such embodiments, the second stent graft is deployed at the desired location and the plunger 168 is threaded through the first stent graft and into the second stent graft to make contact with the first sleeve and push the first sleeve out of the lumen. FIGS. 2A and 2B illustrate such an embodiment. In embodiments utilizing a plunger, a stent graft woven material is woven and oriented to be at least partially resistant to tears from pushing on the material.

Specifically, in FIG. 2A, the stent graft system is illustrated with first stent graft 110 deployed in the thoracic aorta, and second stent graft 120 is illustrated with each of a first sleeve 150 and a second sleeve 160 within the lumen defined by the second stent graft 120. Third stent graft 130 is not illustrated in FIG. 1 for clarity of illustration, although the third stent graft may be deployed while the sleeve 150 is within the lumen in certain embodiments. FIG. 2B then illustrates a plunger 168 within the aorta approaching the sleeves within the lumen. This illustration is exemplary of a femoral approach, although alternate approaches may be clinically desirable depending on patient physiology or other factors, at the discretion of the treating physician. Fluoroscopy may be used to help position plunger 168 to contact the everted end 298 of the sleeve 150 to push the sleeve 150 through itself to deploy sleeve 150 within the exterior of the second stent graft, and within the destination artery. After deployment, the plunger 168 is retracted, and may be used to deploy other sleeves, or the plunger 168 may be simply removed from the vasculature as appropriate.

In another embodiment, the sleeve 150 further includes a loop at a distal end (distal as determined after deployment), and the system includes a hook sized to catch the loop and pull the sleeve 150 from inside the main graft section 155 to outside the main graft section. In such an embodiment, multiple entrances into the vascular system may be required so that the second stent graft can be delivered to the primary vessel via a first approach, while the hook can be delivered into the side vessel to access the sleeve 150 while within the primary vessel and pull the sleeve 150 into the side vessel. Such an embodiment is illustrated, e.g. in FIG. 2C whereby a hook 246 approaches the sleeve 150 and loop 274 to pull the sleeve 150 out of the lumen. In embodiments utilizing the hook and loop, a stent graft woven material is preferred with is at least partially resistant to tears from pulling on the material, and in particular the material used for the sleeve 150 is optimally resistant to pulling forces.

In one embodiment, the first stent graft, second stent graft, and third stent graft are integral with each other. In other embodiments, a two-part system combining either the first stent graft and second stent graft, or the second stent graft and third stent graft, may be utilized. In the modular approach outlined, the need for customized lengths and diameters is reduced as the sizing can be, at least partially, accomplished in the patient. However, it is contemplated that integral stent grafts including a sleeved portion in the middle and a bifurcated distal end may be created.

As also shown in FIGS. 2A, 2B, and 2C, the positioning of the sleeve 150 within the second stent graft 120 can be configured depending on delivery technique as well as deployment method. For example, the sleeve 150 is illustrated as within the second stent graft 120 but with the everted sleeve angled toward the heart, whereas, in FIG. 2B, the everted sleeve 150 is illustrated as angled away from the heart. For example, the angled position away from the heart, as illustrated in FIG. 2B might be preferable when a plunger device is to be used with a femoral insertion, whereas the angled position toward the heart might be preferable for deliveries via an axilliary approach. For example, in FIG. 1, plunger 168 is illustrated in the aortic arch distal the heart, illustrative of the axilliary approach.

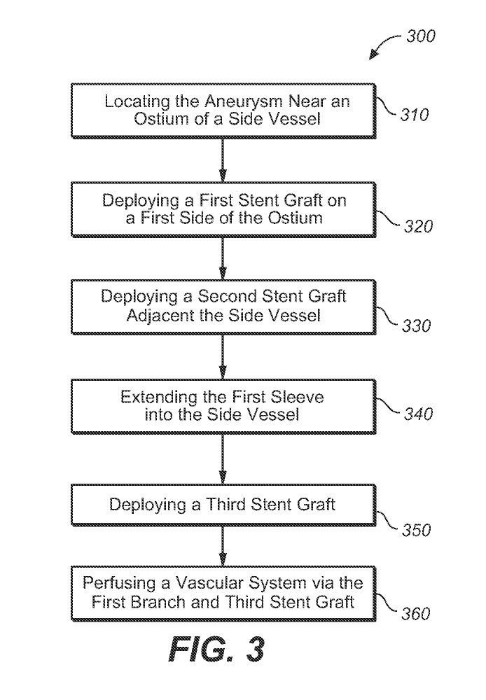

FIG. 3 illustrates the steps of a method 300 for treating an aneurysm, in accordance with one aspect of the invention. Method 300 begins at 310 by locating the aneurysm near an ostium of a side vessel. For example, the main vessel can be the aorta, while the side vessel may be the renal arteries, or celiac arteries or the like. Based on locating the aneurysm to be treated, a first stent graft is deployed on a first side of the ostium at step 320. In most applications, it will be desirable that the distal end of the first stent graft does not obstruct perfusion into the side vessel. After deploying the first stent graft, a second stent graft is deployed at step 330. The second stent graft is deployed adjacent the side vessel based on deploying the first stent graft. The second stent graft is in fluid communication with the first graft and further includes at least a first sleeve extending radially therefrom and in fluid communication with the lumen. In most applications, the second stent graft will be positioned so that the first sleeve is adjacent the ostium. Based on the positioning of the second stent graft, the first sleeve is extended into the side vessel at step 340. The first sleeve can be extended using a plunger device, such as that illustrated in FIG. 2B, or via a hook and loop device, such as illustrated in FIG. 2C. Method 300 continues by deploying a third stent graft at step 350. The third stent graft is in fluid communication with the second stent graft. Method 300 concludes by perfusing a vascular system via the first sleeve and third stent graft at step 360.

Additionally, in certain embodiments of the invention, a therapeutic drug coating may be applied to at least one of the stent graft material and support structure. This therapeutic drug coating can be configured to elute to obtain certain therapeutic goals. Appropriate methods of applying the therapeutic agent include, but are not limited to, dipping, spraying, pad printing, inkjet printing, rolling, painting, micro-spraying, wiping, electrostatic deposition, vapor deposition, epitaxial growth, and combinations thereof. As just one example, the therapeutic agent may be contained in a gel that is sprayed onto the outer surface of the stent framework and the graft member. Exemplary therapeutic drugs include, but are not limited to anti-restenotic drugs, anti-inflammatories, or the like.

Those of skill in the art will recognize that the techniques and structures disclosed herein allow for even a very large aneurysm located near the ostium of a side vessel can be effectively sealed off while maintaining appropriate perfusion through the side vessel as well as downstream from the aneurysm.

While specific embodiments according to the invention are disclosed herein, various changes and modifications can be made without departing from the spirit and scope of the invention.

1. A stent graft system comprising:
a first stent graft having a first end and a second end, the second end opposing the first end;
a second stent graft in fluid communication with the first stent graft, the second stent graft defining a lumen, and including a third end in fluid communication with the second end, a fourth end opposing the third end, and at least a first sleeve extending radially therefrom and in fluid communication with the lumen, and a second sleeve extending radially therefrom and in fluid communication with the lumen, and wherein the first sleeve is radially offset from the second sleeve, wherein the each of the first sleeve and second sleeve comprises a flexible material configured to evert between a first position such that the sleeve is positioned within the lumen and a second position such that the sleeve is positioned exterior to the lumen; and
a third stent graft in fluid communication with the second stent graft, the third stent graft including a fifth end in fluid communication with the fourth end wherein the third stent graft is one of a bifurcated stent graft and an aorto-uni-iliac stent graft.

2. The system of claim 1 further comprising at least a first plunger sized to extend the first sleeve from inside the lumen to the exterior of the lumen.

3. The system of claim 2 further comprising at least a first delivery catheter, and wherein the plunger passes through the delivery catheter to extend the first sleeve.

4. The system of claim 1, wherein the first sleeve includes a loop, and wherein the system further comprises at least a first hook sized to catch the loop and to pull the first sleeve from inside the lumen to the exterior of the lumen, wherein the first hook extends through a vessel in addition to the aorta to catch the loop and pull the first sleeve from inside the lumen.

5. The system of claim 4 wherein the first sleeve includes a proximal portion and a distal portion opposing the proximal portion, and wherein the loop is positioned near the distal portion.

6. The system of claim 1 wherein the first stent graft is a TAA stent graft.

7. The system of claim 1 wherein the sleeve is integral with the second stent graft.

8. A stent graft comprising:
a first portion;
a second portion in fluid communication with the first portion, the second portion defining a lumen, and including a third end in fluid communication with the second end, a fourth end opposing the third end, and at least a first sleeve extending radially therefrom and in fluid communication with the lumen, and a second sleeve extending radially therefrom and in fluid communication with the lumen, and wherein the first sleeve is radially offset from the second sleeve, wherein each of the first sleeve and second sleeve comprises a flexible material configured to evert between a first position such that the sleeve is positioned within the lumen and a second position such that the sleeve is positioned exterior to the lumen; and
a third portion in fluid communication with the second portion, wherein the first portion, second portion, and third portion are integral.

9. The stent graft of claim 8 further comprising at least a first plunger sized to extend the first sleeve from inside the lumen to the exterior of the lumen.

10. The stent graft of claim 9 further comprising at least a first delivery catheter, and wherein the plunger pushes through the delivery catheter to extend the first sleeve.

11. The stent graft of claim 8, wherein the first sleeve includes a loop, and further comprising at least a first hook sized to catch the loop and to pull the first sleeve from inside the lumen to the exterior of the lumen, wherein the first hook extends through a vessel other than the aorta to catch the loop and pull the first sleeve from inside the lumen.

12. The stent graft of claim 11 wherein the first sleeve includes a proximal portion and a distal portion opposing the proximal portion, and wherein the loop is positioned near the distal portion.

13. A method for treating an aneurysm, the method comprising:
locating the aneurysm near an ostium of a side vessel;
deploying a first stent graft on a first side of the ostium;
deploying a second stent graft adjacent the side vessel based on the deploying the first stent graft, wherein the second stent graft is in fluid communication with the first graft, the second stent graft including at least a first sleeve extending radially therefrom and in fluid communication with the lumen, and a second sleeve extending radially therefrom and in fluid communication with the lumen, and wherein the first sleeve is radially offset from the second sleeve, wherein the first sleeve and second sleeve each comprises a flexible material configured to evert between a first position such that the sleeve is positioned within the lumen and a second position such that the sleeve is positioned exterior to the lumen
extending the first sleeve into a side vessel;
extending the second sleeve into a second side vessel; and
deploying a third stent graft, the third stent graft in fluid communication with the second stent graft.

14. The method of claim 13 further comprising stenting the sleeve in place.
